(19) United States
(12) Patent Application Publication
(10) Pub. No.: US 2025/0157625 A1
(43) Pub. Date: May 15, 2025

(54) SYSTEM AND METHOD FOR EVALUATING AND COACHING SOCIAL COMPETENCIES IN IMMERSIVE VIRTUAL ENVIRONMENTS

(71) Applicant: Board of Regents, The University of Texas System, Austin, TX (US)

(72) Inventors: Maria T. Johnson, Austin, TX (US); Aaron M. Tate, Dallas, TX (US)

(73) Assignee: Board of Regents, The University of Texas System, Austin, TX (US)

(21) Appl. No.: 18/414,651

(22) Filed: Jan. 17, 2024

Related U.S. Application Data
(60) Provisional application No. 63/548,341, filed on Nov. 13, 2023.

(51) Int. Cl. G16H 20/70; G10L 15/22; G10L 15/26

(52) U.S. Cl. CPC G16H 20/70; G10L 15/22; G10L 15/26; G10L 2015/225

(57) ABSTRACT

A system for generating objective assessment criteria of social skills, comprising a speech to text system receiving audio data and converting the audio data into text data for a first speaker and a second speaker, a dialog management system processing text data for the first speaker and text data for the second speaker generating a strategic attention score for the first speaker based on responsiveness of the text data of the first speaker to text data of the second speaker, and a response verification system generating a user control for the strategic attention score and receiving feedback data from the second speaker to accept or modify the strategic attention score.

### RELATED APPLICATIONS

This application claims benefit of and priority to U.S. Provisional Patent Application 63/548,341, filed Nov. 13, 2023, which is hereby incorporated by reference for all purposes as if set forth herein in its entirety.

### TECHNICAL FIELD

The present disclosure relates generally to artificial intelligence systems, and more specifically to a system and method for precision brain health assessment that utilizes artificial intelligence to monitor interactions.

### BACKGROUND OF THE INVENTION

When a practitioner interacts with a patient, it can be difficult to assess the entire interaction and to generate objective data that can be used to identify the patient's current status, whether progress has been made with therapy, and where additional work is needed.

### SUMMARY OF THE INVENTION

A system for generating objective assessment criteria of social skills is disclosed. The system includes a speech to text system receiving audio data, such as from participants in a conversation, and converting the audio data into text data for a first speaker and a second speaker. A dialog management system processes text data for the first speaker and text data for the second speaker and generates a strategic attention score for the first speaker based on responsiveness of the text data of the first speaker to text data of the second speaker. A response verification system generates a user control for the strategic attention score and receives feedback data from the second speaker to accept or modify the social competency score.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings may be to scale, but emphasis is placed upon clearly illustrating the principles of the present disclosure. Moreover, in the drawings, like reference numerals designate corresponding parts throughout the several views, and in which:

FIG. 1 is a diagram of a system for performing training and analysis, in accordance with an example embodiment of the present disclosure;

FIG. 2 is a diagram of a system for dialog management, in accordance with example embodiment of the present disclosure;

FIG. 3 is a diagram of a system for a user interface control, in accordance with example embodiment of the present disclosure;

FIG. 4 is diagram of an algorithm for processing weights for an interaction system, in accordance with an example embodiment of the present disclosure;

FIG. 5 is diagram of an algorithm for processing an interaction to identify questions, in accordance with an example embodiment of the present disclosure;

FIG. 6 is diagram of an algorithm for processing an interaction to identify responses, in accordance with an example embodiment of the present disclosure;

FIG. 7 is diagram of an algorithm for processing an interaction to identify a pause, in accordance with an example embodiment of the present disclosure; and

FIG. 8 is diagram of an algorithm for processing an interaction to evaluate questions and responses, in accordance with an example embodiment of the present disclosure.

### DETAILED DESCRIPTION OF THE INVENTION

In the description that follows, like parts are marked throughout the specification and drawings with the same reference numerals. The drawing figures may be to scale and certain components can be shown in generalized or schematic form and identified by commercial designations in the interest of clarity and conciseness.

Artificial intelligence generally refers to a number of different computer systems that can be configured to provide information processing functions to transform inputs into functional outputs. In one example embodiment, a neural network is a type of artificial intelligence that uses an arrangement of nodes to process input data and to generate output data. The output data can be a functional output such as a process control that turns a pump or heater on or off, but artificial intelligence systems are increasingly being used for more complex functions, such as identifying data patterns that are used to fix complex machinery before it fails. In that embodiment, the repair of the equipment might be performed by a human, but the necessity to perform the repair before a catastrophic failure was identified using artificial intelligence, which is thus a functional transformation of the data to provide a functional output that would otherwise not be possible using only human activities.

An essential part of artificial intelligence is training to improve the function of the computer systems. In the example of a neural network, the nodes are trained to process data by receiving inputs and generating an output that is then scored. If the output was wrong, then the nodes are trained to reflect that the output that was generated was incorrect and to provide the correct output. While there are many different training algorithms, neural network algorithms and other artificial intelligence and deep learning algorithms, they all involve data processing and require proper training data to be provided in order to function properly. Thus, the ability to identify specific data for training, such as correct and incorrect outputs for a given input, is critical for the proper functioning of the processors in an artificial intelligence system. An artificial intelligence system that outputs incorrect responses is not just non-operative, but it is also potentially hazardous. For this reason, it is important to have systems for assisting humans with artificial intelligence processing of data that allow humans to independently assess the accuracy of outputs and to provide training data, to improve the functioning of the processors. Because the inputs and outputs of the artificial intelligence system are typically highly specialized, the systems for assisting humans with artificial intelligence processing of data that allow humans to independently assess the accuracy of outputs and to provide training data are also highly specialized for specific applications. The present disclosure provides systems for assisting humans with artificial intelligence processing of psychological counseling data that allow humans to independently assess the accuracy of outputs and to provide training data to the artificial intelligence systems to improve their ability to process data for the purpose of providing psychological counseling.

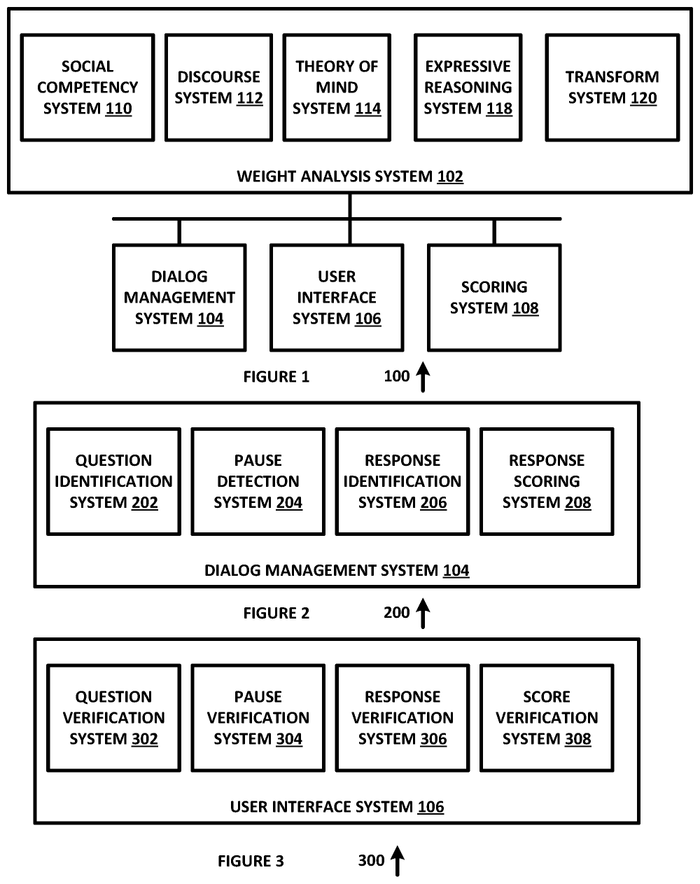

FIG. 1 is a diagram of a system 100 for performing training and analysis, in accordance with an example embodiment of the present disclosure. System 100 includes weight analysis system 102, dialog management system 104, user interface system 106, scoring system 108, social competency system 110, discourse system 112, theory of mind system 114, expressive reasoning system 116 and transform system 118, each of which can be implemented in hardware or suitable combination of hardware and software.

System 100 can be used by a practitioner to evaluate social competency, such as for a wellness check, to treat a patient for social or psychological issues or for other suitable purposes. In one example embodiment, the practitioner can be a psychologist or psychiatrist, and can be treating a patient with a behavioral disorder, such as violent behavior, aggressive behavior, withdrawn behavior, or other behavioral disorders. System 100 provides objective criteria for assessing treatment, such as by establishing weights for a number of factors, including factors relating to social competency, strategic attention, discourse, theory of mind, expressive reasoning, transformational behavior, or other suitable factors. System 100 can use artificial intelligence, deep learning or other suitable analytical systems to transform the interactions between the practitioner and the patient into weighting factors that can be used to assess areas where training or therapy is needed. Weighting factors can start at zero and increase with proficiency, can start at a per unit value of 1 and can be decreased with proficiency, or other suitable weights can also or alternatively be used. In one example embodiment, the practitioner can use a number of online avatars to represent not only themselves but a number of different avatars that each represent different character traits, such as people who are happy, people who are sad, people who are aggressive and so forth, and can interact with the patient to elicit responses to cues, where the responses are analyzed and graded and used to adjust weighting factors for subsequent interactions. In other example embodiments, system 100 can be used for varying levels of immersion, such as to analyze less immersive interactions through text/SMS or social media to find congruent competencies to a lower degree of accuracy. The present disclosure can thus be used to create social profiles that extend beyond the clinical simulation space and have much wider applications.

Weight analysis system 102 can be implemented as one or more lines of code that are stored in a data memory and loaded into a working memory of a processor to cause the processor to be configured to implement an artificial intelligence engine that receives weight data for each of five factors such as strategic attention system 110, discourse system, 112, theory of mind system 114, expressive reasoning system 118, and transform system 120 or other suitable systems, and can adjust weighting for each of a plurality of components. In one example, embodiment, weight analysis system 102 can perform artificial intelligence processing such as neural network training, deep learning training, or other suitable training to adjust weights based on inputs from dialogue management system, 104, user interface, 106 and scoring system 108 or other suitable inputs.

Dialogue management system 104 can be implemented as one or more lines of code that are stored in a data memory and loaded into a working memory of a processor to cause the processor to be configured to implement an artificial intelligence engine that receives dialogue that has been converted from speech to text data, and can process the dialogue to identify questions, answers, speakers, pauses, and other suitable dialogue metadata. In other example embodiments, real-time tonal quality, facial expression analysis or other suitable data can also or alternatively be analyzed.

User interface system 106 can be implemented as one or more lines of code that are stored in a data memory and loaded into a working memory of a processor to cause the processor to be configured to implement an artificial intelligence engine that processes questions, pause data, response data, score data and other suitable data, and generates a user interface prompt to allow a user to identify whether or not questions, pauses, responses, scores, or other suitable data have been properly generated. Likewise, a user can identify questions, pauses, responses, scores that were missed by the artificial intelligence systems, can adjust scores or other suitable data, and can perform other suitable functions.

Scoring system 108 can be implemented as one or more lines of code that are stored in a data memory and loaded into a working memory of a processor to cause the processor to be configured to implement an artificial intelligence engine that receives question and answer data and generates a score, such as by using an artificial intelligence process that determines whether an answer to a question is compliant with expected answers, is not compliant, falls somewhere between compliant and non-compliant, or otherwise meets certain scoring criteria. Scoring system 108 can generate score data for presentation to user interface system 106, can receive updated score information from user interface system 106 that can be used to adjust the training used by scoring system 108 to generate scores, and can perform other suitable functions.

Strategic attention system 110 can be implemented as one or more lines of code that are stored in a data memory and loaded into a working memory of a processor to cause the processor to be configured to implement an artificial intelligence engine that receives data and determines whether it relates to strategic attention criteria for grading or other purposes. In one example embodiment, strategic attention criteria can include sustained virtual immersion analysis factors, where on-target social behaviors for these factors include whether the patient attends to elements of the virtual world, whether the patient shows curiosity by exploring the environment or moving within the virtual space, whether the patient is aware and responsive to avatars and the environment, or other suitable factors. In contrast, off-target social behaviors can include the patient “running away” in the virtual environment, the patient being distracted by voices, graphics or gameplay in the virtual environment, the patient switching out of the virtual world to use a different software applications, the patient multitasking or otherwise making it difficult for the practitioner to interact with the patient. On-target and off-target responses and social behaviors can be used to train the artificial intelligence systems, but application of specific interactions to on-target and off-target weights will typically require additional training. For example, if attending to elements of the virtual world is an on-target behavior, the virtual world can have a number of elements, where some are more important than others. For example, an avatar walking into a virtual room can have a greater importance than an object representing a piece of furniture in that room, depending on the context. Training is used to improve the function of artificial intelligence systems to apply interactions to on-target and off-target weights and to modify those weights when needed.

In another example embodiment, strategic attention criteria can include attention to nonverbal cues and emotions factors, where on-target social behaviors by the patient include noticing the practitioner's thoughts, feelings, needs or overt actions, whether the patient picks up on most relevant/important conversational details to stay engaged, whether the patient directs attention towards the practitioner more than environment or other suitable factors. Off-target social behaviors can include long pauses, no response or unrelated responses, not enquiring about the practitioner, hyper-focus on objects rather than the practitioner or other factors.

In another example embodiment, strategic attention criteria can include whether the patient can read a situation accurately. In this example embodiment, on-target social behaviors can include whether the patient forms accurate impressions given the context, whether the patient has expected reactions to the practitioner's mood, vibe, energy or emotions given the scenario, whether the patient maintains an expected level of intensity during the interaction or other suitable factors. Off-target social behaviors can include whether the patient forms snap judgements, makes conversation uncomfortable, creates negative or inaccurate assumptions or other suitable factors.

Discourse system 112 can be implemented as one or more lines of code that are stored in a data memory and loaded into a working memory of a processor to cause the processor to be configured to implement an artificial intelligence engine that receives data and determines whether it relates to discourse capability criteria for grading or other purposes. In one example embodiment, discourse system 112 can evaluate whether the patient is able to maintain expected reciprocity, where on-target social behaviors include the ability to initiate communications or responses to the practitioner, the ability to maintain a conversational flow with back-and-forth conversational turns, the ability to use simple responses or ask basic questions, the ability to make related statements using simple 3-to-5-word phrases or exchanges or other suitable factors. Off-target social behaviors include talking can over the practitioner or interrupting, silences that disrupt the overall flow of conversation, overuse of very basic responses, such as one or two word responses, overuse of conversational fillers, such as um, uh and so forth.

In another example embodiment, discourse capability criteria can include whether the patient can share thoughts, opinions and experiences, where on-target social behaviors include relating to the practitioner by sharing the patients ideas or experiences that are on-topic, providing personal details about hobbies, likes, dislikes or opinions, sharing statements of the patients own feelings and emotions that relate to conversations and so forth. Off-target social behaviors include over-sharing personal information, asking too many questions, providing surface-level or superficial responses, such as “ok,” “cool,” “interesting” and the like, sharing but not allowing for conversational turns at expected intervals and so forth.

In another example embodiment, discourse capability criteria can include whether the patient can shift to or with new topics, where on-target social behaviors include following the practitioner's lead when a topic is changed, staying engaged without confusion, asking follow-up questions and sharing thoughts on new topic, developing a deeper connection by showing interest in multiple topics, having conversational depth or allows for a breadth of topic discussion and so forth. Off-target social behaviors include becoming stuck, confused, or lost with new topics, not inquiring, following up with or moving along with new topics, making awkward, unrelated, or unexplained topic shifts, reverting back to preferred topics despite the practitioners efforts to change the topic and so forth.

Theory of mind system 114 can be implemented as one or more lines of code that are stored in a data memory and loaded into a working memory of a processor to cause the processor to be configured to implement an artificial intelligence engine that receives data and determines whether it relates to theory of mind criteria for grading or other purposes. In one example embodiment, theory of mind criteria can include the ability to recognize different perspectives, where on-target social behaviors include considering that the practitioner has their own point of view that is separate, and possibly different from the patient's own point of view, understanding that the practitioner's reactions may be unique to and potentially dependent on their preexisting knowledge or experiences, acknowledging differences in viewpoints and so forth. Off-target social behaviors can include only considering the patient's own perspective, not realizing or disregarding that the practitioner is also forming impressions and thoughts, not acknowledging responding and/or reacting to differing perspectives, providing responses that demonstrate a general lack of awareness that the patient and practitioner have mismatched ideas, and so forth.

In another example embodiment, theory of mind criteria can include the ability to perceive covert intentions or motives, where on-target social behaviors include reading indirect or implied social cues accurately and adapting response accordingly, asking follow-up questions to gain clarity or additional details, reacting with caution, care or concern, and so forth. Off-target social behaviors include requiring direct questions in order to expand or inquire further, requiring overt explanations in order to respond to the conversational context, not indicating an awareness of social concepts such as rumors, scams, deception and so forth.

In another example embodiment, theory of mind criteria can include the ability to identify potential problems and outcomes, where on-target social behaviors include mentioning or alluding to a problem or likely outcome related to the conversational context, suggesting or advising at least one prosocial and safe idea or solution, providing or arriving at an integrated “ah-ha” moment, using rules/laws/authority expectations as a way of responding to or framing the situation and so forth. Off-target social behaviors include remaining quiet when a problem is presented, not showing any indication of thinking ahead, not mentioning or show an integrated understanding of how actions or choices may be perceived by or affect others, not behaving in a prosocial and safe manner and so forth.

Expressive reasoning system 118 can be implemented as one or more lines of code that are stored in a data memory and loaded into a working memory of a processor to cause the processor to be configured to implement an artificial intelligence engine that receives data and determines whether it relates to expressive reasoning criteria for grading or other purposes. In one example embodiment, expressive reasoning criteria can include the ability to generate multiple solutions or alternative possibilities, where on-target social behaviors include evaluating ideas regarding what to do next or instead when initial suggestion is ineffective, responding with more than the initial option or choice, generating at least two solutions or ideas for each dilemma presented, attempting to negotiate, persuade, or compromise in a prosocial manner that is neither detrimental to the patient nor practitioner and so forth. Off-target social behaviors can include not offering multiple ideas or solutions, repeating the same idea over and over, trying to force the patient's own perspective on the practitioner in a detrimental manner and so forth.

In another example embodiment, expressive reasoning criteria can include the ability to communicate cohesively and concisely, where on-target social behaviors include connecting important information between or across conversations and practitioner personas as necessary, selectively choosing information to share that is helpful and that is easy to follow along, actively navigating within a conversation to arrive at the point with ease and sensitivity, interpreting or summarizing to relay or express information and so forth. Off-target social behaviors include fragmented or partial statements, overly blunt or abrupt responses, responses that include unhelpful information, using he-said-she-said recall/retell, getting easily lost in the weeds, lacking story organization, providing details that bounce around a lot or that are too frequently modified and hard to follow, and so forth.

In another example embodiment, expressive reasoning criteria can include the ability to explain thinking and decision making, where on-target social behaviors can include explaining what could causally happen (e.g. “if-then”) and “why” it is salient remaining prosocial and safe with the added or matters, requirements of being believable and realistic, offering deeper level meaning or learnings that shows critical thinking, using disclaimers, repairing miscommunications, providing others with insight into actions, decisions, or choices and so forth. Off-target social behaviors include providing irrelevant, unjustified, or stereotyped responses that are not prosocial, responding only with “because” or “I don't know,” a minimal ability to expand or provide justification, relying too heavily on citing laws, rules or authority expectations and so forth.

Transform system 120 can be implemented as one or more lines of code that are stored in a data memory and loaded into a working memory of a processor to cause the processor to be configured to implement an artificial intelligence engine that receives data and determines whether it relates to transformational criteria for grading or other purposes. In one example embodiment, transformational criteria can include adapting to situational needs, where on-target social behaviors include pro-socially adjusting an approach, trying new ways to respond, remaining calm, centered and agile, responding nonjudgmentally to the situation with overt assertion, empathy, curiosity, caution, or flexibility, depending on context, changing an approach swiftly, decisively, and appropriately based on new information layered in by the practitioner and so forth. Off-target social behaviors can include flight, fight, or freeze responses that prevent adapting, not changing or incorporating new information into a response, pushing an agenda regardless of new information or changes in plans and so forth.

In another example embodiment, transformational criteria can include responding resiliently to mishaps or hardship, where on-target social behaviors include responding resourcefully and with savviness, recovering quickly with confidence or commitment to morals, beliefs and values, reframing personal difficulties or obstacles as personal growth opportunities, breaking the status quo, embracing change, focusing on making ecological choices that result in a positive impact on self, others, and the environment or group, and so forth. Off-target social behaviors can include criticizing self or others, being unable to bounce back from the unexpected or from emotional responses, a misalignment between a reaction to a problem and the significance of the problem, expressing what self or others “should” do, being stuck in the mistake or past experiences and so forth.

In operation, system 100 allows a practitioner to engage with a patient to perform an evaluation of the patient's mental states. Because mental states are often internal, it is necessary to have a highly structured input and output processing structure to evaluate and obtain objective data. In this manner, system 100 transforms subjective states into objective criteria that can be used for further evaluation.

FIG. 2 is a diagram of a system 200 for dialog management, in accordance with example embodiment of the present disclosure. System 200 includes dialog management system 104 and question identification system 202, pause detection system 204, response identification system 206 and response scoring system 208, each of which can be implemented in hardware or suitable combination of hardware and software.

Question identification system 2022 can be implemented as one or more lines of code that are stored in a data memory and loaded into a working memory of a processor to cause the processor to be configured to implement an artificial intelligence engine that receives text data that has been converted from speech and can determine whether a question has or has not been presented. In one example embodiment, a speech to text translator can be used and can include meta-data that identifies speech characteristics of a speaker. Question identification system 202 can use the converted speech data, the meta-data and other suitable data to determine whether a question was posed by a practitioner, a patient, a third-party, or other suitable persons. Question identification system 202 can also generate a prompt for verification of the question by the practitioner or other suitable persons, where the verification data can be used to train an artificial intelligence, deep learning or other suitable system to improve its ability to identify questions.

Pause detection system 204 can be implemented as one or more lines of code that are stored in a data memory and loaded into a working memory of a processor to cause the processor to be configured to implement an artificial intelligence engine that receives text data that has been converted from speech and can determine whether a speaker has or has not paused. In one example embodiment, a speech to text translator can be used and can include meta-data that identifies speech characteristics of a speaker. Pause detection system 204 can use the converted speech data, the meta-data and other suitable data to determine whether a speaker has paused and whether the speaker is a practitioner, a patient, a third-party, or other suitable persons. Pause detection system 204 can also generate a prompt for verification of whether a pause occurred by the practitioner or other suitable persons, where the verification data can be used to train an artificial intelligence, deep learning or other suitable system to improve its ability to identify pauses.

Response identification system 206 can be implemented as one or more lines of code that are stored in a data memory and loaded into a working memory of a processor to cause the processor to be configured to implement an artificial intelligence engine that receives text data that has been converted from speech and can determine whether a response to a question has or has not been presented. In one example embodiment, a speech to text translator can be used and can include meta-data that identifies speech characteristics of a speaker. Response identification system 206 can use the converted speech data, the meta-data and other suitable data to determine whether a response to a question was posed by a practitioner, a patient, a third-party, or other suitable persons has been received. Response identification system 206 can also generate a prompt for verification of the response to a question by the practitioner or other suitable persons, where the verification data can be used to train an artificial intelligence, deep learning or other suitable system to improve its ability to identify responses.

Response scoring system 208 can be implemented as one or more lines of code that are stored in a data memory and loaded into a working memory of a processor to cause the processor to be configured to implement an artificial intelligence engine that receives text data that has been converted from speech and can determine a score for a response to a question. In one example embodiment, a speech to text translator can be used and can include meta-data that identifies speech characteristics of a speaker. Response scoring system 208 can use the converted speech data, the meta-data and other suitable data to score a response to a question was posed by a practitioner, a patient, a third-party, or other suitable persons. Response scoring system 208 can also generate a prompt for verification of the score by the practitioner or other suitable persons, where the verification data can be used to train an artificial intelligence, deep learning or other suitable system to improve its ability to score responses to questions.

FIG. 3 is a diagram of a system 300 for a user interface control, in accordance with example embodiment of the present disclosure. System 300 includes user interface system 106 and question verification system 302, pause verification system 304, response verification system 306 and score verification system 308, each of which can be implemented in hardware or suitable combination of hardware and software.

Question verification system 302 can be implemented as one or more lines of code that are stored in a data memory and loaded into a working memory of a processor to cause the processor to be configured to implement an artificial intelligence engine that receives text data that has been converted from speech and can determine whether a question has or has not been verified. In one example embodiment, a prompt for verification of the question by the practitioner or other suitable persons can include a number of additional tools to allow the user to modify a question to make it accurate, to withdraw the question so it is not used for training, or to provide other suitable input. The verification data can be used to train an artificial intelligence, deep learning or other suitable system to improve its ability to identify questions, such as by editing the data that was used by the artificial intelligence, deep learning or other system to generate the identified question to include more or less data, to change a weight for certain words associated with the question or in other suitable manners.

Pause detection system 304 can be implemented as one or more lines of code that are stored in a data memory and loaded into a working memory of a processor to cause the processor to be configured to implement an artificial intelligence engine that receives text data that has been converted from speech and can determine whether a pause has or has not been verified. In one example embodiment, a prompt for verification of the pause by the practitioner or other suitable persons can include a number of additional tools to allow the user to modify a pause to make it accurate, to withdraw the question or other statement associated with the pause so it is not used for training, or to provide other suitable input. In this example embodiment, if a pause is preceded by someone stating “I'll be back in a minute,” then the pause should not be identified as an improper pause, and the artificial intelligence engine used for pause detection system 304 can be trained to recognize that and other appropriate pauses and to distinguish them from inappropriate pauses. The verification data can be used to train an artificial intelligence, deep learning or other suitable system to improve its ability to identify a pause, such as by editing the data that was used by the artificial intelligence, deep learning or other system to identify a pause to include more or less data, to change a weight for certain words associated with the pause or in other suitable manners.

Response verification system 306 can be implemented as one or more lines of code that are stored in a data memory and loaded into a working memory of a processor to cause the processor to be configured to implement an artificial intelligence engine that receives text data that has been converted from speech and can determine whether a response has or has not been verified. In one example embodiment, a prompt for verification of the response by the practitioner or other suitable persons can include a number of additional tools to allow the user to modify a question to make it accurate, to withdraw the question so it is not used for training, or to provide other suitable input. In this example, a first person could say “I'll be with you in a moment” after a second person asks a question like “what is your favorite color,” where it would be understood that the first person was speaking to someone else and that the statement should not be processed as a response to the question. The verification data can be used to train an artificial intelligence, deep learning or other suitable system to improve its ability to identify responses, such as by editing the data that was used by the artificial intelligence, deep learning or other system to generate the identified response to include more or less data, to change a weight for certain words associated with the response or in other suitable manners.

Score verification system 308 can be implemented as one or more lines of code that are stored in a data memory and loaded into a working memory of a processor to cause the processor to be configured to implement an artificial intelligence engine that receives text data that has been converted from speech and can determine whether a score has or has not been verified. In one example embodiment, a prompt for verification of the score by the practitioner or other suitable persons can include a number of additional tools to allow the user to modify a question or response associated with a score to make it accurate, to withdraw the question and/or response so it is not used for training, or to provide other suitable input. The verification data can be used to train an artificial intelligence, deep learning or other suitable system to improve its ability to score responses to questions, such as by editing the data that was used by the artificial intelligence, deep learning or other system to generate the score to include more or less data, to change a weight for certain words associated with the score or in other suitable manners.

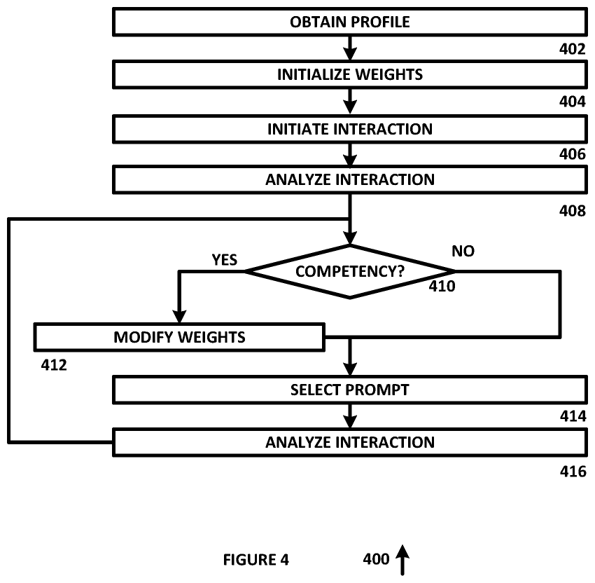

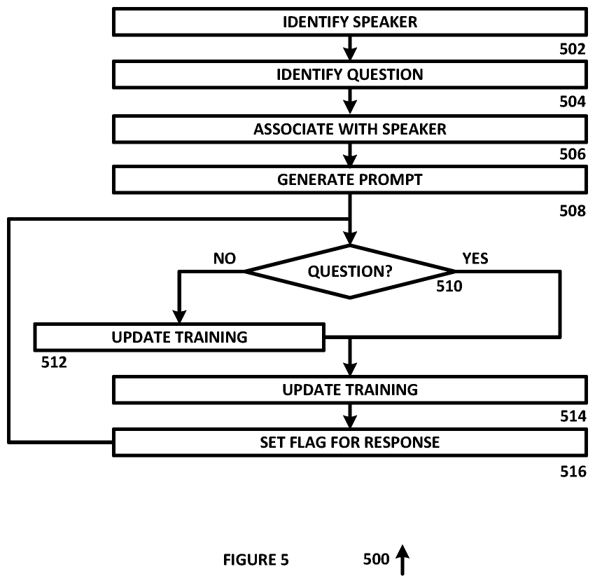

FIG. 4 is diagram of an algorithm 400 for processing weights for an interaction system, in accordance with an example embodiment of the present disclosure. Algorithm 400 can be implemented in hardware or suitable combination of hardware and software.

Algorithm 400 begins at 402, where a profile is obtained. In one example embodiment, the profile can be retrieved from a profile database, the profile can be entered by user, or other suitable processes can also or alternatively be used. The algorithm then proceeds to 404.

At 404, a plurality of weights are initialized for the profile. In one example embodiment, the weights can correspond to factors for strategic social attention and associated subcomponents, discourse and associated subcomponents, theory of mind and associated subcomponents, expressive reasoning and associated subcomponents, transformation and associated subcomponents and other suitable categories. The algorithm then proceeds to 406.

At 406, an interaction is initiated. In one example embodiment, the interaction can be initiated by a practitioner starting a session with a patient. The interaction can include one or more virtual environment elements, such as where a practitioner is controlling a number of avatars, where an avatar controlled by a third party, a script or an artificial intelligence engine is used, where a virtual environment activity is generated, or other suitable interactions can be initiated. The algorithm then proceeds to 408.

At 408, the interaction is analyzed. In one example embodiment, speech data can be converted to text data and associated metadata, such as data that identifies a speaker, an amount of elapsed time, speech patterns that reflect strong emotional content such as rhythm and inflections, and so forth. The interaction can be continuously analyzed to evaluate whether questions, responses, pauses, or other suitable functional attributes have been identified in the interaction. The algorithm then proceeds to 410.

At 410, it is determined whether a competency has or has not been expressed. In one example embodiment, text data can be processed using an artificial intelligence process, a deep learning process or other suitable processes to determine whether a competency has or has not been expressed, such as by processing the text data and metadata to determine whether it can be associated with a competency. In one example embodiment, for strategic social attention, a competency can be expressed if a patient exhibits on-target social behavior for sustaining virtual immersion by attending to elements of a virtual environment. In this example embodiment, the text data can include a statement like “an antelope just ran by” when an antelope runs by in the virtual environment, and where the associated metadata includes data identify that an event consisting of an antelope running by has occurred. In this example, the competency of exhibiting on-target social behavior for sustaining virtual immersion by attending to elements of a virtual environment would have been exhibited, and the algorithm would proceed to 412. Likewise, if the antelope ran by 5 minutes before the statement, the competency determination for sustaining virtual immersion by attending to elements of a virtual environment would not be considered to have been met, and the algorithm would proceed to 414. Other suitable analyses can also or alternatively be performed to evaluate whether additional or different competencies have been exhibited, as discussed further herein.

At 412, weights associated with the competency are adjusted to reflect that the patient should be considered to have acquired a higher level of competency, such as to assess whether training can be considered completed, whether a more difficult test should be administered, or whether other suitable functional transformations should be implemented. In one example embodiment, the weights can be adjusted by increasing the weight if a competency has been demonstrated, by decreasing the weight if the competency has not been demonstrated prior to proceeding from 410 to 414, or other suitable processes can also alternatively be used.

At 414, a prompt is selected. In one example embodiment, the prompt can include a request for a practitioner to confirm a weight modification, to confirm a competency or failure to exhibit a competency, or to confirm other suitable indications. The algorithm then proceeds to 416.

At 416, an interaction occurs and is analyze. In one example embodiment, the interaction can include a subsequent verbal interaction with or without interaction with the virtual environment or other suitable interactions. The algorithm then returns to 410.

In operation, algorithm 400 allows a practitioner to interact with a patient in a structured manner, so as to generate objective data that can be used to analyze the patient's state of mind and cognitive processing. In this manner, objective data regarding how a patient interacts with other and performs cognitive processing can be obtained and used to evaluate the patient for treatment.

FIG. 5 is diagram of an algorithm 500 for processing an interaction to identify questions, in accordance with an example embodiment of the present disclosure. Algorithm 500 can be implemented in hardware or suitable combination of hardware and software.

Algorithm 500 begins at 502, where a speaker is identified. In one example embodiment, speech data can be converted to text data and associated metadata, such as data that identifies a speaker, an amount of elapsed time, speech patterns that reflect strong emotional content such as rhythm and inflections, and so forth. The interaction can be continuously analyzed to evaluate whether questions, responses, pauses, or other suitable functional attributes have been identified in the interaction. The algorithm then proceeds to 504.

At 504, a question is identified. In one example embodiment, the speech data and associated metadata can be analyzed using an artificial intelligence system, a deep learning system or other suitable systems that use algorithms that are trained to identify whether a question has or has not been asked, such as by analyzing a first text string for a first speaker, a second text string for a second speaker that immediately preceded the first text string, speech inflection metadata, environment relevance metadata (such as the text string “what is that” being spoken immediately after an object or avatar appears on screen) or in other suitable manners. The algorithm then proceeds to 506.

At 506, the question is associated with a speaker. In one example embodiment, the speaker can be identified using frequency characteristics of audio data and voice identification data that was previously obtained, the speaker can be identified using audio source identifiers or the speaker can be identified in other suitable manners. The algorithm then proceeds to 508.

At 508, a prompt is generated. In one example embodiment, the prompt can be an on-screen prompt at a user interface of the practitioner that requests the practitioner to confirm whether a question was or was not asked, and can include the converted speech to text string or other suitable data. The algorithm then proceeds to 510.

At 510, it is determined whether a question has or has not been asked. In one example embodiment, the practitioner can select a control that indicates that the identified text string was (e.g. “ACCEPT”) or was not (e.g. “REJECT”) a question, the practitioner can modify the text string to correct any errors in the speech to text conversion (e.g. “MODIFY”) or other suitable modifications or controls can be activated. If a question has been asked, the algorithm proceeds to 514, otherwise the algorithm proceeds to 512.

At 512, training is updated to reflect an incorrect identification of a question. In one example embodiment, the artificial intelligence system, deep learning system or other suitable system that is used to process converted speech data and metadata can include a feedback control for indicating whenever an output is incorrect, has been modified or to provide other learning or feedback data. The algorithm then proceeds to 514.

At 514, the training is updated to reflect the correct question. In one example embodiment, the artificial intelligence system, deep learning system or other suitable system that is used to process converted speech data and metadata can include a feedback control for indicating whenever an output is correct, for processing a modified corrected identification of a question or to provide other learning or feedback data. The algorithm then proceeds to 516.

At 516, a flag is set for a response. In one example embodiment, the response can be generated by the practitioner, by an artificial intelligence system, by a deep learning system or in other suitable manners. The algorithm then returns to 510.

In operation, algorithm 500 is used to identify and process questions for use in analyzing an interaction between a practitioner and a patient or for other suitable processes. Although algorithm 500 is shown as a flow chart, a person of skill in the art will recognize that it can also or alternatively be implemented using object-oriented programming, a state diagram, a ladder diagram, using a combination of programming paradigms or in other suitable manners.

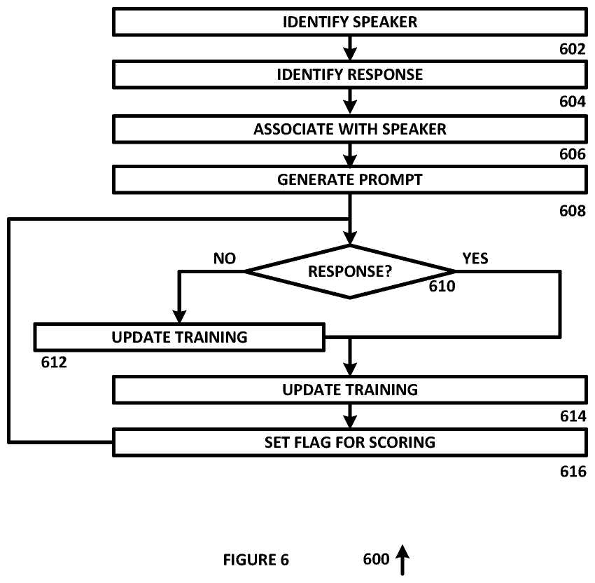

FIG. 6 is diagram of an algorithm 600 for processing an interaction to identify responses, in accordance with an example embodiment of the present disclosure. Algorithm 600 can be implemented in hardware or suitable combination of hardware and software.

Algorithm 600 begins at 602, where a speaker is identified. In one example embodiment, speech data can be converted to text data and associated metadata, such as data that identifies a speaker, an amount of elapsed time, speech patterns that reflect strong emotional content such as rhythm and inflections, and so forth. The interaction can be continuously analyzed to evaluate whether questions, responses, pauses, or other suitable functional attributes have been identified in the interaction. The algorithm then proceeds to 604.

At 604, a response to a question is identified. In one example embodiment, the speech data and associated metadata can be analyzed using an artificial intelligence system, a deep learning system or other suitable systems that use algorithms that are trained to identify whether a response has or has not been provided to a question that was asked, such as by analyzing a first text string for a first speaker, a second text string for a second speaker that immediately followed the first text string, speech inflection metadata, environment relevance metadata (such as the text string “what is that” being spoken immediately after an object or avatar appears on screen) or in other suitable manners. The algorithm then proceeds to 606.

At 606, the response is associated with a speaker. In one example embodiment, the speaker can be identified using frequency characteristics of audio data and voice identification data that was previously obtained, the speaker can be identified using audio source identifiers or the speaker can be identified in other suitable manners. The algorithm then proceeds to 608.

At 608, a prompt is generated. In one example embodiment, the prompt can be an on-screen prompt at a user interface of the practitioner that requests the practitioner to confirm whether a response was provided to a question that was asked, and can include the converted speech to text string or other suitable data. The algorithm then proceeds to 610.

At 610, it is determined whether a response to a question has or has not been received. In one example embodiment, the practitioner can select a control that indicates that the identified text string was or was not a response to a question, the practitioner can modify the text string to correct any errors in the speech to text conversion or other suitable modifications or controls can be activated. If a response to a question has been provided, the algorithm proceeds to 614, otherwise the algorithm proceeds to 612.

At 612, training is updated to reflect an incorrect identification of a response to a question. In one example embodiment, the artificial intelligence system, deep learning system or other suitable system that is used to process converted speech data and metadata can include a feedback control for indicating whenever an output is incorrect, has been modified or to provide other learning or feedback data. The algorithm then proceeds to 614.

At 614, the training is updated to reflect the correct identification of a response to the question. In one example embodiment, the artificial intelligence system, deep learning system or other suitable system that is used to process converted speech data and metadata can include a feedback control for indicating whenever an output is correct, for processing a modified corrected identification of a response to a question or to provide other learning or feedback data. The algorithm then proceeds to 616.

At 616, a flag is set for a response. In one example embodiment, the response can be generated by the practitioner, by an artificial intelligence system, by a deep learning system or in other suitable manners. The algorithm then returns to 610.

In operation, algorithm 600 is used to identify and process responses to questions for use in analyzing an interaction between a practitioner and a patient or for other suitable processes. Although algorithm 600 is shown as a flow chart, a person of skill in the art will recognize that it can also or alternatively be implemented using object-oriented programming, a state diagram, a ladder diagram, using a combination of programming paradigms or in other suitable manners.

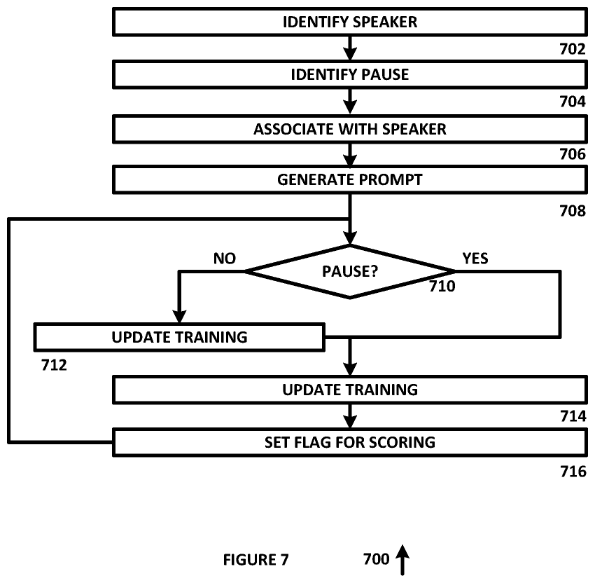

FIG. 7 is diagram of an algorithm 700 for processing an interaction to identify a pause, in accordance with an example embodiment of the present disclosure. Algorithm 700 can be implemented in hardware or suitable combination of hardware and software.

Algorithm 700 begins at 702, where a speaker is identified. In one example embodiment, speech data can be converted to text data and associated metadata, such as data that identifies a speaker, an amount of elapsed time, speech patterns that reflect strong emotional content such as rhythm and inflections, and so forth. The interaction can be continuously analyzed to evaluate whether questions, responses, pauses, or other suitable functional attributes have been identified in the interaction. The algorithm then proceeds to 704.

At 704, a pause is identified. In one example embodiment, the speech data and associated meta data can be analyzed using an artificial intelligence system, a deep learning system or other suitable systems that use algorithms that are trained to identify whether a speaker has paused, such as by analyzing a first text string for a first speaker, a second text string for a second speaker that immediately preceded the first text string, speech inflection metadata, environment relevance metadata (such as the text string “what is that” being spoken immediately after an object or avatar appears on screen) or in other suitable manners. The algorithm then proceeds to 706.

At 706, the pause is associated with a speaker. In one example embodiment, the speaker can be identified using frequency characteristics of audio data and voice identification data that was previously obtained, the speaker can be identified using audio source identifiers or the speaker can be identified in other suitable manners. The algorithm then proceeds to 708.

At 708, a prompt is generated. In one example embodiment, the prompt can be an on-screen prompt at a user interface of the practitioner that requests the practitioner to confirm whether a pause occurred, and can include the converted speech to text string or other suitable data. The algorithm then proceeds to 710.

At 710, it is determined whether a pause has or has not occurred. In one example embodiment, the practitioner can select a control that indicates that the identified text string contained a pause, the practitioner can modify the text string to correct any errors in the speech to text conversion or other suitable modifications or controls can be activated. If a pause has occurred, the algorithm proceeds to 714, otherwise the algorithm proceeds to 712.

At 712, training is updated to reflect an incorrect identification of a pause. In one example embodiment, the artificial intelligence system, deep learning system or other suitable system that is used to process converted speech data and metadata can include a feedback control for indicating whenever an output is incorrect, has been modified or to provide other learning or feedback data. The algorithm then proceeds to 714.

At 714, the training is updated to reflect a correct pause. In one example embodiment, the artificial intelligence system, deep learning system or other suitable system that is used to process converted speech data and metadata can include a feedback control for indicating whenever an output is correct, for processing a modified corrected identification of a pause or to provide other learning or feedback data. The algorithm then proceeds to 716.

At 716, a flag is set for a response. In one example embodiment, the response can be generated by the practitioner, by an artificial intelligence system, by a deep learning system or in other suitable manners. The algorithm then returns to 710.

In operation, algorithm 700 is used to identify and process pauses for use in analyzing an interaction between a practitioner and a patient or for other suitable processes. Although algorithm 700 is shown as a flow chart, a person of skill in the art will recognize that it can also or alternatively be implemented using object-oriented programming, a state diagram, a ladder diagram, using a combination of programming paradigms or in other suitable manners.

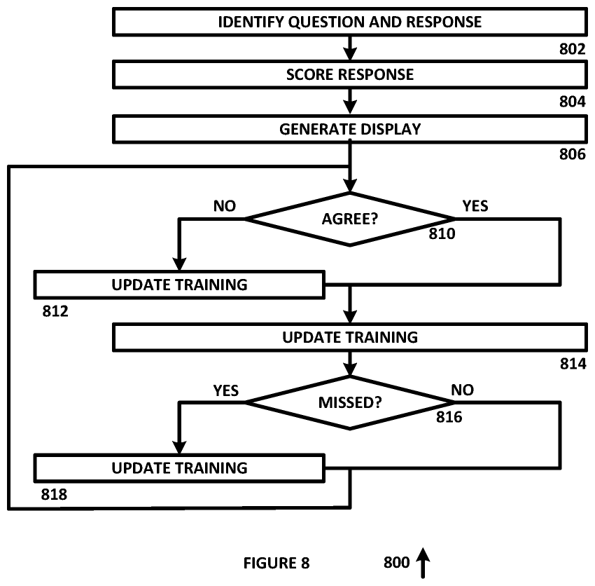

FIG. 8 is diagram of an algorithm 800 for processing an interaction to evaluate questions and responses, in accordance with an example embodiment of the present disclosure. Algorithm 800 can be implemented in hardware or suitable combination of hardware and software.

Algorithm 800 begins at 802, where speakers are identified. In one example embodiment, speech data can be converted to text data and associated metadata, such as data that identifies a speaker, an amount of elapsed time, speech patterns that reflect strong emotional content such as rhythm and inflections, and so forth. The interaction can be continuously analyzed to evaluate whether questions, responses, pauses, or other suitable functional attributes have been identified in the interaction. The algorithm then proceeds to 804.

At 804, the question and associated response are scored. In one example embodiment, the score can utilize predefined on-target and off-target social behaviors for a plurality of factors, such as for strategic social attention and associated subcomponents, discourse and associated subcomponents, theory of mind and associated subcomponents, expressive reasoning and associated subcomponents, transformation and associated subcomponents and other suitable categories. The question and response can be analyzed using artificial intelligence systems, deep learning systems or other suitable systems that have bene trained to identify questions and responses associated with those factors, scores associated with the questions and responses and other suitable data. The algorithm then proceeds to 806.

At 806, a display is generated that presents the question, the response, the score, the factor, the on-target criteria, the off-target criteria and other suitable data for review by a practitioners. In one example embodiment, the display can provide the practitioner with sufficient information perform an independent assessment of the score, to evaluate whether they agree with the score, need to modify the score or if other actions need to be taken. The algorithm then proceeds to 808.

At 808, a prompt is generated to allow the practitioner to indicate whether they agree with the score. In one example embodiment, the prompt can be an on-screen prompt at a user interface of the practitioner that requests the practitioner to confirm whether the proposed score is correct, and can include the converted speech to text string or other suitable data. If the practitioner does not agree, the algorithm then proceeds to 810 where the training for the artificial intelligence system, deep learning system or other suitable system is updated. In one example embodiment, a feedback can be provided to indicate whether an output is correct or incorrect, to provide a corrected output or to provide other suitable data. The algorithm then proceeds to 812.

At 812, the training for the artificial intelligence system, deep learning system or other suitable system is updated. In one example embodiment, a feedback can be provided to indicate whether an output is correct or incorrect, to provide a corrected output or to provide other suitable data. The algorithm then proceeds to 814.

At 814, it is determined whether a question and response was missed by the artificial intelligence system, deep learning system or other suitable system. If it is determined that a question and response was not missed, the algorithm returns to 802. If it is determined that a question and response was missed, the algorithm proceeds to 816 where training for the system is implemented, such as by providing the text string for the missed question and answer, an associated score and other suitable data. The algorithm then returns to 802.

In one example embodiment, a feedback can be provided to indicate whether an output is correct or incorrect, to provide a corrected output or to provide other suitable data. The algorithm then proceeds to 812. training is updated to reflect an incorrect identification of a question. In one example embodiment, the artificial intelligence system, deep learning system or other suitable system that is used to process converted speech data and metadata can include a feedback control for indicating whenever an output is incorrect, has been modified or to provide other learning or feedback data.

In operation, algorithm 800 is used to process an interaction to evaluate questions and responses or for other suitable processes. Although algorithm 800 is shown as a flow chart, a person of skill in the art will recognize that it can also or alternatively be implemented using object-oriented programming, a state diagram, a ladder diagram, using a combination of programming paradigms or in other suitable manners.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes one or more microcomputers or other suitable data processing units, memory devices, input-output devices, displays, data input devices such as a keyboard or a mouse, peripherals such as printers and speakers, associated drivers, control cards, power sources, network devices, docking station devices, or other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections. The term “data” can refer to a suitable structure for using, conveying or storing data, such as a data field, a data buffer, a data message having the data value and sender/receiver address data, a control message having the data value and one or more operators that cause the receiving system or component to perform a function using the data, or other suitable hardware or software components for the electronic processing of data.

In general, a software system is a system that operates on a processor to perform predetermined functions in response to predetermined data fields. A software system is typically created as an algorithmic source code by a human programmer, and the source code algorithm is then compiled into a machine language algorithm with the source code algorithm functions, and linked to the specific input/output devices, dynamic link libraries and other specific hardware and software components of a processor, which converts the processor from a general purpose processor into a specific purpose processor. This well-known process for implementing an algorithm using a processor should require no explanation for one of even rudimentary skill in the art. For example, a system can be defined by the function it performs and the data fields that it performs the function on. As used herein, a NAME system, where NAME is typically the name of the general function that is performed by the system, refers to a software system that is configured to operate on a processor and to perform the disclosed function on the disclosed data fields. A system can receive one or more data inputs, such as data fields, user-entered data, control data in response to a user prompt or other suitable data, and can determine an action to take based on an algorithm, such as to proceed to a next algorithmic step if data is received, to repeat a prompt if data is not received, to perform a mathematical operation on two data fields, to sort or display data fields or to perform other suitable well-known algorithmic functions. Unless a specific algorithm is disclosed, then any suitable algorithm that would be known to one of skill in the art for performing the function using the associated data fields is contemplated as falling within the scope of the disclosure. For example, a message system that generates a message that includes a sender address field, a recipient address field and a message field would encompass software operating on a processor that can obtain the sender address field, recipient address field and message field from a suitable system or device of the processor, such as a buffer device or buffer system, can assemble the sender address field, recipient address field and message field into a suitable electronic message format (such as an electronic mail message, a TCP/IP message or any other suitable message format that has a sender address field, a recipient address field and message field), and can transmit the electronic message using electronic messaging systems and devices of the processor over a communications medium, such as a network. One of ordinary skill in the art would be able to provide the specific coding for a specific application based on the foregoing disclosure, which is intended to set forth exemplary embodiments of the present disclosure, and not to provide a tutorial for someone having less than ordinary skill in the art, such as someone who is unfamiliar with programming or processors in a suitable programming language. A specific algorithm for performing a function can be provided in a flow chart form or in other suitable formats, where the data fields and associated functions can be set forth in an exemplary order of operations, where the order can be rearranged as suitable and is not intended to be limiting unless explicitly stated to be limiting.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A system for generating objective assessment criteria of social skills, comprising:
a speech to text system operating on a processor and configured to receive audio data and to convert the audio data into text data for a first speaker and a second speaker;
a dialog management system operating on the processor and configured to process text data for the first speaker and text data for the second speaker to generate a social competency score for the first speaker based on responsiveness of the text data of the first speaker to text data of the second speaker; and
a response verification system operating on the processor and configured to generate a user control for the social competency score and to receive feedback data from the second speaker to accept or modify the social competency score, wherein the text data of the first speaker, the text data of the second speaker, the social competency score data and the feedback data is use to train an artificial intelligence system to improve recognition of responsiveness of the text data of the first speaker to text data of the second speaker.

2. The system of claim 1 wherein the dialog management system is configured to generate the social competency score using a weight analysis system that is configured to apply one or more weights to the text data of the first speaker and the text data of the second speaker.

3. The system of claim 1 wherein the dialog management system is configured to generate the social competency score using a strategic attention weight analysis system that is configured to apply one or more strategic attention weights to the text data of the first speaker and the text data of the second speaker to generate a strategic attention score.

4. The system of claim 1 wherein the dialog management system is configured to generate the social competency score using a discourse weight analysis system that is configured to apply one or more discourse weights to the text data of the first speaker and the text data of the second speaker to generate a discourse score.

5. The system of claim 1 wherein the dialog management system is configured to generate the social competency score using a theory of mind weight analysis system that is configured to apply one or more theory of mind weights to the text data of the first speaker and the text data of the second speaker to generate a theory of mind score.

6. The system of claim 1 wherein the dialog management system is configured to generate the social competency score using an expressive reasoning weight analysis system that is configured to apply one or more expressive reasoning weights to the text data of the first speaker and the text data of the second speaker to generate an expressive reasoning score.

7. The system of claim 1 wherein the dialog management system is configured to generate the social competency score using a transform weight analysis system that is configured to apply one or more transform weights to the text data of the first speaker and the text data of the second speaker to generate a transform score.

8. The system of claim 1 wherein the dialog management system is configured to generate the social competency score using a plurality of weight analysis systems that are each configured to apply one or more weights to the text data of the first speaker and the text data of the second speaker to generate an associated score profile.

9. The system of claim 1 wherein the dialog management system is configured to generate the social competency score using a plurality of weight analysis systems that are each configured to apply one or more weights to the text data of the first speaker and the text data of the second speaker to generate an associated score.

10. The system of claim 9 wherein the scores for the plurality of weight analysis systems are used to generate proposed interactions between the first speaker and the second speaker.

11. A method for generating objective assessment criteria of social skills, comprising:
receiving audio data at a speech to text system operating on a processor;
converting the audio data into text data for a first speaker and a second speaker;
processing text data for the first speaker and text data for the second speaker using a dialog management system operating on the processor to generate a social competency score for the first speaker based on responsiveness of the text data of the first speaker to text data of the second speaker;
generating a user control for the social competency score using a response verification system operating on the processor;
receiving feedback data from the second speaker to accept or modify the social competency score; and
wherein the text data of the first speaker, the text data of the second speaker, the social competency score data and the feedback data is use to train an artificial intelligence system to improve recognition of responsiveness of the text data of the first speaker to text data of the second speaker.

12. The method of claim 11 further comprising generating the social competency score using a weight analysis system that is configured to apply one or more weights to the text data of the first speaker and the text data of the second speaker.

13. The method of claim 11 further comprising generating the social competency score using a strategic attention weight analysis system that is configured to apply one or more strategic attention weights to the text data of the first speaker and the text data of the second speaker to generate a strategic attention score.

14. The method of claim 11 further comprising generating the social competency score using a discourse weight analysis system that is configured to apply one or more discourse weights to the text data of the first speaker and the text data of the second speaker to generate a discourse score.

15. The method of claim 11 further comprising generating the social competency score using a theory of mind weight analysis system that is configured to apply one or more theory of mind weights to the text data of the first speaker and the text data of the second speaker to generate a theory of mind score.

16. The method of claim 11 further comprising generating the social competency score using an expressive reasoning weight analysis system that is configured to apply one or more expressive reasoning weights to the text data of the first speaker and the text data of the second speaker to generate an expressive reasoning score.

17. The method of claim 11 further comprising generating the social competency score using a transform weight analysis system that is configured to apply one or more transform weights to the text data of the first speaker and the text data of the second speaker to generate a transform score.

18. The method of claim 11 further comprising generating the social competency score using a plurality of weight analysis systems that are each configured to apply one or more weights to the text data of the first speaker and the text data of the second speaker to generate an associated score profile.

19. The method of claim 11 further comprising generating the social competency score using a plurality of weight analysis systems that are each configured to apply one or more weights to the text data of the first speaker and the text data of the second speaker to generate an associated score.

20. The method of claim 19 wherein the scores for the plurality of weight analysis systems are used to generate proposed interactions between the first speaker and the second speaker.
